(19) United States
(12) Patent Application Publication
(10) Pub. No.: US 2006/0194231 A1
(43) Pub. Date: Aug. 31, 2006

(54) Suspension arrays of cross-reactive oligonucleotide-based sensors

(76) Inventors: Milan N. Stojanovic, Fort Lee, NJ (US); Sergei Rudchenko, Bronx, NY (US)

Correspondence Address: COOPER & DUNHAM, LLP, 1185 AVENUE OF THE AMERICAS, NEW YORK, NY, 10036, US

(21) Appl. No.: 11/340,145

(22) Filed: Jan. 25, 2006

Related U.S. Application Data
(60) Provisional application No. 60/646,719, filed on Jan. 25, 2005.

(51) Int. Cl. C12Q 1/68

(52) U.S. Cl. 435/6

(57) ABSTRACT

Cross-reactive arrays on encoded beads are used to correlate ‘fingerprints’ of urine, serum and other biological liquids to disease states. Fluorescent hydrophobic sensors are based on nucleic acid three-way junctions, and beads may be encoded by size (could be registered by light scattering) and fluorescence in combination with flow cytometry analysis, also known as suspension array technology (SAT).

### STATEMENT REGARDING FEDERALLY SPONSORED RESEARCH OR DEVELOPMENT

The work discussed herein was supported by NIH grant NBIB R01-EB 000675-01 and NASA grant NS2-02039. The U.S. government may have rights to this invention.

### BACKGROUND OF THE INVENTION

Throughout this application, various publications are referenced to as footnotes or within parentheses. Disclosures of these publications in their entireties are hereby incorporated by reference into this application to more fully describe the state of the art to which this invention pertains. Full bibliographic citations for these references may be found at the end of this application, preceding the claims.

The mammalian olfactory system consists of approximately one thousand receptors, expressed in up to one hundred million cells (Axel 1995). The distinctive characteristic of this system is cross-reactivity, i.e. one receptor may react with many odorants, and one odorant may react with many receptors. Thus, an odorant is not characterized with a single and specific interaction, but rather through a pattern of massively parallel responses yielding a fingerprint characteristic for that specific odorant. Attempts to mimic the mammalian olfactory system have led to the development of “electronic noses”, or arrays of cross-reactive sensors (Albert 2000, Lavigne 2001, Schauer 2001, Rakow 2000). However, the types of biomolecular sensors that can be incorporated into cross-reactive arrays for solution applications are currently limited. One likely reason is the traditional view that the true value of biomolecular receptors (i.e. antibodies and oligonucleotide-based aptamers) is in their high specificity. Accordingly, cross-reactivity in complex mixtures is usually viewed as detrimental. The other reason is the lack of frameworks suitable for the incremental variations of structures necessary to achieve differential cross-reactivity. The third possible reason is lack of general ability to turn biomolecular receptors into sensors that could be used in a single-step, mix-and-measure assays.

Flow cytometry is a process in which one performs the measurement of multiple physical characteristics of individual particles (traditionally cells, thus the name) (Nolan 1999). In flow cytometry, particles in a fluid stream cross one or more laser beams and the corresponding light scattering (i.e. particle size and internal complexity) and fluorescent emission from fluorophores are being registered by multiple detectors. This technology is capable of making sensitive (a few hundred to a few thousand fluorescent molecules) and quantitative measurements of several different fluorescent probes simultaneously on individual particles. Flow cytometry is mainly used for quantitative analysis of cell populations, based on multiparameter fluorescence. One may employ microspheres labeled by up to two different fluorochromes and also by the size to unambiguously encode different sensors in the array. In suspension arrays individual elements are identified by one or more intrinsic optical properties of a particle (bead or microsphere) in suspension. A 64-element suspension array (8×8, i.e. eight different intensities for each of two fluorescent dyes contained within microspheres) has been recently reported (Kettman 1998), opening the possibility for up to 64 sensors to be used in an array, that was extended up to 100-element suspension array (10×10, i.e. for up to 100 sensors to be used in an array) by Luminex Corp.

### SUMMARY OF THE INVENTION

According to the invention, one may use crossreactive receptors or sensors productively in cross-reacting suspension arrays. Each of the guest molecules could interact with more than one receptor or sensor. Thus, this system may be used to determine whether several could form an array capable of generating fingerprints characteristic for hydrophobic compounds, and thereby create a primitive solution-phase mimic of the olfactory system. This technology is proving to be compatible with a range of assay chemistries in a high-throughput format. In suspension arrays individual elements are identified by one or more intrinsic optical properties of a particle (bead or microsphere) in suspension.

The invention provides a paradigm-shifting approach to mix-and measure solution-phase diagnostics and the ability to move away from traditional sensor approaches based on aptamers and antibodies. More specifically: (1) one may construct a cross-reactive suspension array for analysis of, for example, hydrophobic molecules, in which a group of sensors will be organized on coded beads, and show that quantitative measurement of fluorescence by flow cytometry analysis of these beads will yield a characteristic profile for solution; (2) this technique may improve the sensitivity of such arrays by up to 100-fold; (3) the fingerprints of a solution grossly deviating from the clinical norm could be easily correlated to a clinical condition; (4) A closer mimicking of the resolution power of mammalian olfactory sense may be obtained by incorporating in array elements up to 100 of closely related, yet distinct, sensors; (5) the successful development of the first nucleic acid-based cross-reacting arrays for hydrophobic fingerprinting could provide an impetus for the examination of other cross-reactive suspension nucleic acid-based arrays, for which no comparable methods exist (e.g., for monitoring fine variations in blood and urinary oligosacharides and glycopeptide glycoforms); (6) the successful design of suspension arrays based on cross-reactive DNA sensors will inspire theoretical and computer-assisted modeling approaches, which will lead to an increased understanding of the principles behind recognition of hydrophobic small molecules by hydrophobic cavities in general and, nucleic acids, in particular.

The invention provides the capability to improve human health through improved detection and diagnosis of diseases. Specifically, one would expect to develop cross-reactive suspension arrays for analytes, which were traditionally much too complex to analyze routinely.

According to the invention, a suspension cross-reactive array for detecting the presence of at least one analyte is provided, comprising a plurality of particles each encoded by a different selected characteristic, a plurality of cross-reactive sensors, wherein at least one sensor is responsive to the presence of more than one different analyte, and wherein at least two sensors are responsive to the same analyte, wherein each particle is attached to a different sensor, and wherein the identification of the analyte is detected by the arrangement of encoding of the particles' characteristics.

A method for detecting the presence of at least one analyte is provided, comprising providing a plurality of particles each encoded by a different selected characteristic, providing a plurality of cross-reactive sensors, wherein at least one sensor is responsive to the presence of more than one different analyte, wherein at least two sensors are responsive to the same analyte, and wherein each particle is attached to a different sensor, detecting the identification of the analyte by the arrangement of encoding of the particles' characterstics.

### DESCRIPTION OF THE DRAWINGS

FIG. 1. A. Schematic representation of the sensors based on three-way junction with a single phosphorothioate, that is derivatized with fluorophore (F). Black ellipsoid represents hydrophobic molecule that upon binding displaces fluorophore, causing an increase in fluorescence (larger font). Only one phosphorothioate isomer is shown. B. Sensor based on a mismatched junction recognizes ligand and signals this recognition through increase in fluorescence of a displaced fluorophore.

FIG. 2. Fingerprints of four ligands: cocaine 1-500 μM, deoxycorticosterone 21-glucoside 2-32 μM, dehydroisoandrosterone 3-sulfage 3-125 μM, and sodium deoxycholate 4-2 mM with an array of seven sensors, represented as an increase in fluorescence after the addition of ligand to individual wells containing sensors. Each pattern is a response to one of the sensors, seven patterns together represent fingerprint of an analyte.

FIG. 3. Fingerprints of urine (U), urine spiked with deoxycorticosterone 21-glucoside 2 (U+2) and urine spiked with dehydroisoandrosterone 3-sulfate 3 (U+3) black: 4.1-7F8; white: fmtch-A23-32F33; dark gray: fmtch-T25-32F33; light-gray: 4.1-32F33. Triplicate measurements of fluorescence intensity were taken, with standard deviation shown.

FIG. 4. Concentration dependence of deoxycorticosterone 21-glucoside on fluorescence intensity of microspheres with attached sensors. A. Relative changes of mean fluorescence of conjugated beads (5000 events per point). Value 100% is corresponding to difference between mean fluorescence of conjugated beads in solution without analyte and autofluorescence of unconjugated beads. B. Examples of frequency distributions of microspheres. Each histogram represent 5000 beads.

FIG. 5. The structures of two junctions first for possible separation and attachment to beads. 4.1-32F33 was tested on beads. The structure of representative ligands: cocaine (1), deoxycorticosterone 21-glucoside (2), dehydroisoandrosterone 3-sulfate (3) and deoxycholic acid (4).

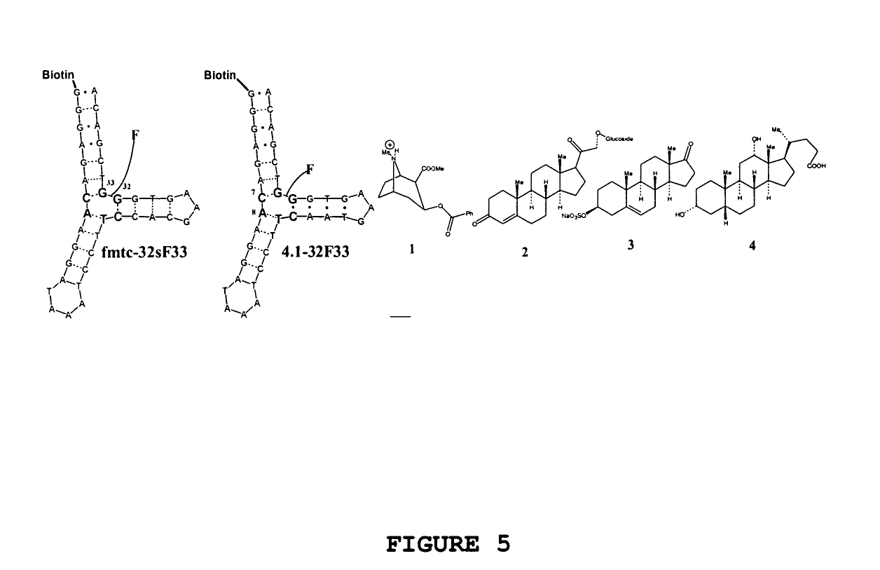

FIG. 6. A sample of modifications of junctions introducing variability in the hydrophobic shapes of the junction.

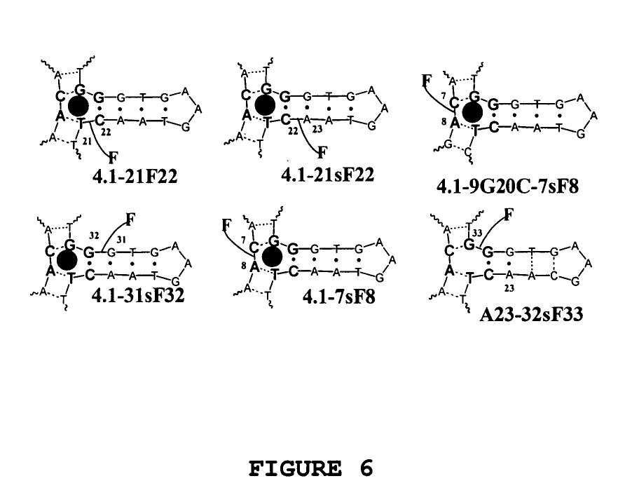

FIG. 7. Computer simulation of fingerprint pattern of five-element suspension array as example of data representation. Ordinate (Y axis) dot plot represents encoding of beads. Abscissa (X axis)—fluorescent signal from sensors.

FIG. 8. Examples of modified bases used to generalize approach to cross-reactive arrays: 1 and 3 may be used to construct recognition elements for sugars, 2 may be used to construct recognition elements for lipids, while 4 may be used to recognize peptides and to study phosphorylation patterns.

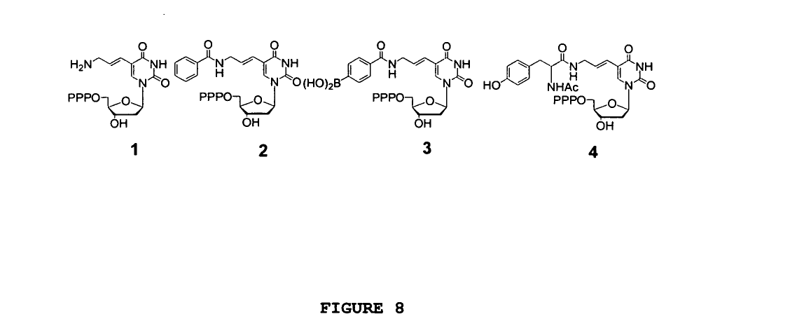

### DESCRIPTION OF THE INVENTION

According to the invention, a suspension cross-reactive array for detecting the presence of at least one analyte is provided, comprising a plurality of particles each encoded by a different selected characteristic, a plurality of cross-reactive sensors, wherein at least one sensor is responsive to the presence of more than one different analyte, and wherein at least two sensors are responsive to the same analyte, wherein each particle is attached to a different sensor, and wherein the identification of the analyte is detected by the arrangement of encoding of the particles' characteristics.

The selected characteristic may be size and/or fluorescence. The cross-reactive sensors may comprise oligonucleotides. The sensors may give fluorescent signals. The decoding characteristics of particles and signals from sensors, attached to particles, may be detected simultaneously from each particle. The identification of the analyte may be detected using flow cytometry.

A method for detecting the presence of at least one analyte is provided, comprising providing a plurality of particles each encoded by a different selected characteristic, providing a plurality of cross-reactive sensors, wherein at least one sensor is responsive to the presence of more than one different analyte, wherein at least two sensors are responsive to the same analyte, and wherein each particle is attached to a different sensor, detecting the identification of the analyte by the arrangement of encoding of the particles' characterstics.

The selected characteristic may be size and/or fluorescence. The cross-reactive sensors may comprise oligonucleotides. The sensors may give fluorescent signals. The identification of the analyte may be detected by detecting the arrangement of encoding of the beads' characteristics simultaneously. The identification of the analyte may be detected using flow cytometry.

Nucleic acid junctions are formed at the intersection of three and more double helixes. One may isolate and characterize the first cocaine-binding three-way junctions with mismatched stems (Stojanovic 2000, 2001, 2002). The incorporation of hydrophobic molecules into various nucleic acid junctions may be noticed during early footprinting studies on these structures (Kato 2000). These observations have been confirmed by the isolation of anti-steroid nucleic acid receptors or aptamers that were based on fully matched three-way junctions. The three exposed aromatic surfaces of unstacked base-pairs in three-way junctions form a lipophilic cavity, approximately 11 Å in diameter, which is capable of binding numerous hydrophobic guest molecules.

One unique characteristic and an important advantage of junction receptors is the ease with which the structure of the receptor can be systematically varied through introduction of mutations, mismatches and chemical modifications. According to a preliminary screening, any one of the junctions tested could interact, with different strengths, with multiple guest molecules. Each of the guest molecules could interact with more than one junction. Thus, this system may be used to determine whether several sensors based on these junctions could form an array capable of generating fingerprints characteristic for hydrophobic compounds, and thereby create a primitive solution-phase mimic of the olfactory system.

Following on the seminal work of Ueno and colleagues on cyclodextrines, one may determine whether introduction of a fluorophore into the hydrophobic cavity of the junction would yield a molecular sensor based on the internal displacement of the fluorophore by a guest molecule (Ikeda 1996). Eight out of ten fluorescent junctions have been found to behave like sensors. These were used to obtain characteristic fingerprints for four representative hydrophobic molecules, in a proof-of-concept array in microtiter plates. Based on this, one may continue systematic screening of junctions and to pursue the full characterization of the steroidal hydrophobic space.

The cross-reactive arrays can be expanded to microprint sensors on slides; however, one may focus on a related suspension array technology (Nolan 2001, 2002). This technology is proving to be compatible with a range of assay chemistries in a high-throughput format. Our choice of this technology for practical applications of our cross-reactive arrays is based on good availability of flow cytometry instruments in almost all hospitals, rapid time of analysis and expected excellent sensitivity of this technology. Oligonucleotide-based arrays may be used for diagnostic, therapeutic and computation purposes.

The work described herein includes the small scale synthesis and massive initial screening of oligonucleotide-based sensors as diastereomeric mixtures, as well as the collecting of samples for clinical trials with sensors on microplates. Proof-of-concept arrays capable of detecting steroids have been developed. Further work covers the large-scale synthesis of sensors suitable for attachment to beads and testing of the concept of cross-reactive arrays applied to a flow cytometry set-up.

A class of DNA-based receptors or sensors can be rationally and systematically varied in structure. Some of these receptors can be adapted to yield molecular scale sensors and an array of these sensors can provide a fingerprint for hydrophobic molecules analogous to identification by olfaction. Furthermore, these sensors remain fully functional upon attachment to solid surfaces. Construction of arrays of sensors on encoded beads can be used in combination with a flow cytometry protocol (suspension array technology). Cross-reactive arrays of up to 10 different sensors can be used to test to fingerprint steroidal content in urine. This approach can be expanded to other analytes and suspension arrays of up to 100 different sensors. The invention may provide synthesis and purification of sensors for coupling on beads, construction of cross-reactive suspension arrays on encoded beads, correlation of fingerprints of urine on beads with disease states and therapeutic regimen, and expansion of this approach to oligosaccharides and their conjugates, lipids and peptides. One may work to obtain fingerprints of the errors in glycosylation patterns of peptides and oligosaccharide metabolism. This would allow construction of arrays, which could be routinely and cost-effectively used for neonatal screening of rare inborn diseases.

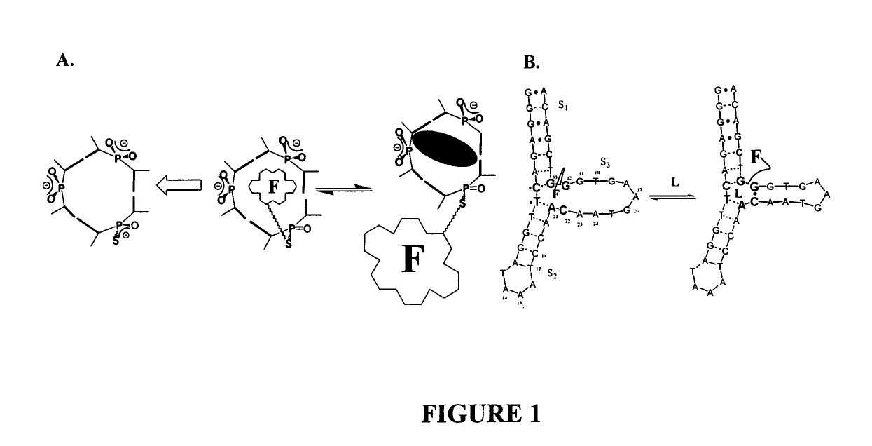

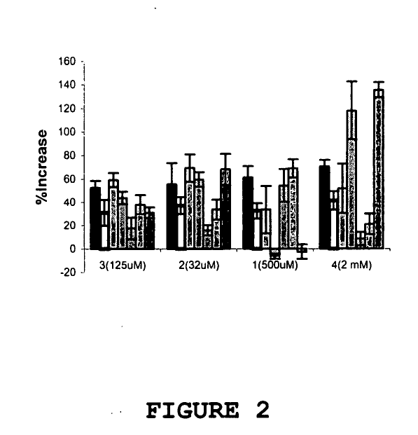

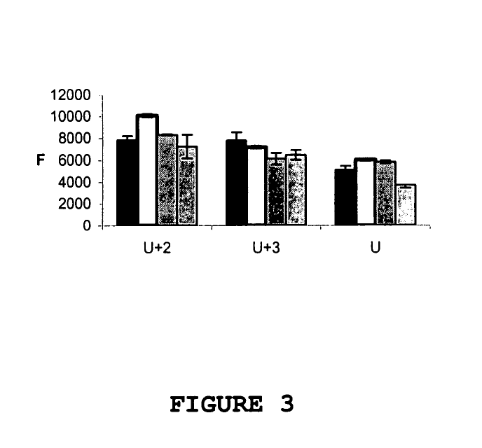

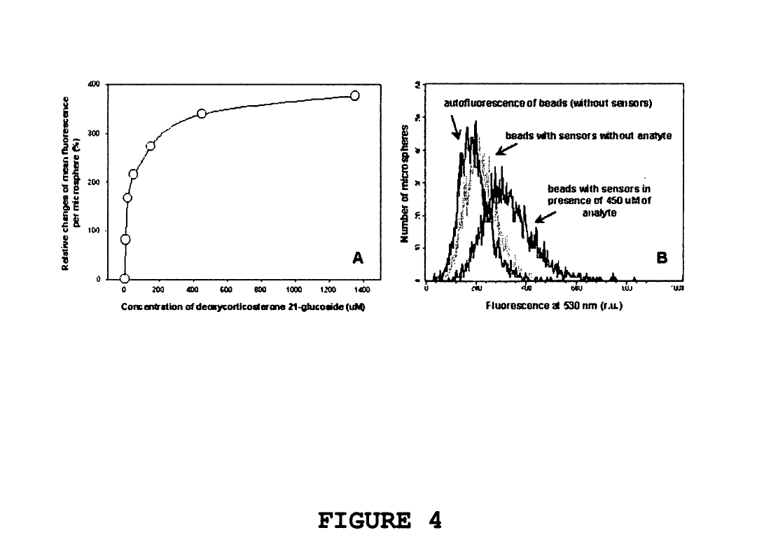

Our earlier work in the area of nucleic acid arrays includes the following:
1. Development of the first general method to convert oligonucleotide-based receptors into sensors (Stojanovic et al. J. Am. Chem. Soc. 2000). Anti-cocaine and anti-ATP aptamers were converted into two oligonucleotide chains that self-assembled in the presence of their ligands. Fluorescence quenching was used to determine ligand concentrations. The two sensors operated independently in solution, and this was used to demonstrate multicolor detection. In unpublished results this method was extended to proteins (thrombin) and a tri-color detection.
2. Development of folding sensors (Stojanovic et al. J. Am. Chem. Soc. 2001). The folding of oligonucleotide-based receptors around their ligands can be used to construct molecular sensors. This method was demonstrated with a hydrophobic molecule (cocaine) and a polar molecule (ATP).
3. Construction of the first colorimetric sensors based on displacement of chromophores from hydrophobic pocket (Stojanovic & Landry J. Am. Chem. Soc, 2002). A potentially general approach to colorimetric sensors based on aptamers was developed.
4. Internal displacement sensors based on hydrophobic pockets of three-way junctions (Stoianovic et al. in press, J. Am. Chem. Soc. 2003). A two-step method was adapted for the sensor construction, in which a single phosphorothioate group was introduced in an aptamer, followed by the selective functionalization of this group with a thiol-reactive fluorophore (Fidanza 1992). As a result, a mixture of diastereomeric sensors was obtained, in which concentrations of hydrophobic molecules in solution were obtained. (FIG. 1)
5. A proof-of-concept of DNA-based array of cross-reactive hydrophobic DNA receptors (Stojanovic et al. J. Am. Chem. Soc. 2003). An array of seven sensors was used to test four ligands at concentrations that gave similar results for a single sensor. The array clearly and reproducibly distinguished the solutions (FIG. 2).
6. Sensor array can unambiguously distinguish fingerprints of urine from urine spiked with steroids (Stojanovic, et al. J. Am. Chem. Soc. 2003). Fingerprints of urine (U), urine spiked with deoxycorticosterone 21-glucoside 2 (U+2) and urine spiked with dehydroisoandrosterone 3-sulfate 3 (U+3) were obtained. (FIG. 3)
7. Biotin-modified junction sensors could be attached to streptavidine coated beads, and could report concentration of steroids in flow-cytometry (Stojanovic & Rudchenko, unpublished results). Fluorescent signal provided by sensors in this system strongly correlates with the concentration of analytes in solution. (FIG. 4)
8. Catalytic oligonucleotides were studied for as sensors (Stojanovic et al Nucleic Acids Res. 2000, Chem. Bio. Chem. 2001). Arrays of nucleic acid sensors, either stoichiometric or catalytic, are believed to be capable of more complex behavior than could be represented through a simple sum of the behaviors of individual sensors. A biomimetic approach to intelligent sensor arrays capable of performing Boolean calculations of arbitrary complexity in solution has resulted in the following.

Overall, initial studies demonstrated the versatility of stoichiometric and catalytic sensors based on nucleic acids, and their capability to behave in a complex manner when organized in arrays. We have also clearly demonstrated the inadequacy of the existing technologies that use in vitro selection and amplification to isolate aptamers as the first step to obtain fluorescent molecular sensors; the limited structural motifs (i.e. mostly unstacked bases and base-pairs) result in the inability to produce significant specificity for hydrophobic molecules. This lack of specificity is a general problem in recognition of hydrophobic molecules by both synthetic and biomolecular receptors, and makes them applicable only within certain contexts (e.g. ultra-high throughput screening in controlled environment). A similar problem exists in the analytical methods based on nucleic acids for determination of oligosaccharides, short oligopeptides and glucopeptides. For example, while moderate selectivity for binding to the targeted disaccharide has been reported earlier, the structural similarity of these molecules would dictate significant cross-reactivity. One may generate cross-reactive sensors for other groups of analytes. The resulting sensors may used on beads, as well.

The invention provides cross-reactive arrays on encoded beads that can be used to correlate ‘fingerprints’ of urine, serum and other biological liquids to disease states. One may use fluorescent hydrophobic sensors based on nucleic acid three-way junctions, and beads may be encoded by size (could be registered by light scattering) and fluorescence in combination with flow cytometry analysis, also known as suspension array technology (SAT).

One may screen up to 3,000 three- and four-way junction-based sensors. The sensor may be synthesized as diastereomeric mixtures at phosphorous and will be used as such directly in arrays in microplate readers and on microchips. One may construct additional oligonucleotides, which are cross-reactive with different types of molecules, e.g. lipids, saccharides and peptides. One may select 6-12 sensors from this effort for attachment to beads. These sensors be synthesized on a larger scale (100 mg) with biotin (or amine) modifications, and the two diastereomers may be separated by an affinity column in HPLC.

One may accomplish standardization of fingerprints. One may observe minor batch-to-batch variations in the diastereomeric content of each sensor. This variation would not significantly influence the gross fingerprints in solution, i.e. each batch would be able to distinguish urine samples from spiked urine samples. However, in initial testing of bead sensors this variation would influence the magnitude and sensitivity of response. As the hydrophobic fingerprints on a given array are an intrinsic characteristic of each hydrophobic compound and mixture, conceptually similar to IR patterns, through the process of scale-up and purification one would avoid the necessity of the separate standardization of each batch. Furthermore, one would: (1) provide sufficient material to complete clinical trials; (2) be able to share with the biosensor community procedures that would guarantee reproducible inter-lab fingerprints; (3) have sufficient material to commence initial studies on attaching these materials to optical fibers; (4) demonstrate that diastereomerically pure sensors have a significantly higher response to analytes, because in many cases only one diastereomer is responsive; and (5) provide material for structural characterization of the mechanism of action for the sensors.

One may have sensor precursor junction with single phosphorothioate bond (cf. FIG. 1) custom synthesized on 100 mg scale. Through small scale testing one may first optimize the reaction with 6-IAF to achieve the highest conversion of the starting material and the best HPLC yield of two diastereomers. One may vary co-solvents, temperatures, amount of 6-IAF and concentration of reactants. The optimized conditions may then be applied on successively larger scale (1, 10, 20, 70 mg scale). Batches may be combined and HPLC purified using an affinity column.

The affinity column used previously for the separation of diastereomers may be made of cocaine attached to agarose. The hydrophobic pocket of an initial junction MNS4.1 interacts strongly with cocaine, and individual diastereomers containing fluorophore interacted differently with cocaine (Kd's˜1 mM and 0.2 mM, respectively). However, this cocaine-based affinity material will not interact with fully-matched junctions and will be easily degraded under physiological pH. Therefore, one may construct two affinity materials, the first based on quaternary ammonium salts and the second based on corticosterone. Our preliminary results show that quaternary and tertiary ammonium salts interact strongly with 90%+ of negatively charged junctions. Some neutral junctions and ˜10% of negatively charged junctions (especially with the fluorophore especially at T21FC22 position) interacted poorly with ammonium salts; but these could be purified with corticosterone-base columns. Concentration of quaternary salts on this affinity column should be low, because high concentrations would affect the ion-exchange mechanism instead of desired affinity purification. The two affinity approaches will allow one to achieve a single diastereomer purity of higher than 90+%. The second round of purification may be performed on standard C18 column, if necessary, in order to separate any non-diastereomeric materials which co-elute with sensors. One would expect to obtain at least 40 mg of each diastereomer at the end of this process, and this amount of material should be sufficient for at least 10,000 samples (an (under)estimation based on preliminary results with mixtures of diastereomers). Importantly, one could separately test both diastereomers of each sensor, and in some cases both isomers may act as sensors. Finally, some material may be used for NMR studies of the sensors to reveal the mechanism at work in these sensors. Details at the molecular level may provide additional inspiration for new and improved sensors. The purification procedure may be performed for each sensor selected for suspension arrays.

One would expect to be able to prepare and test affinity columns within a matter of months while later one may have at least six sensors prepared for the construction of the first array.

Construction of Cross-Reactive Arrays

Before large-scale synthesis and purification all selected sensors should be tested for activity on beads as diastereomeric mixtures. This will insure that only sensors with proper activity would synthesize on a larger scale. One may first use biotinylated oligonucleotides with streptavidine coated polystyrene beads. Alternatively, one may use amine-derivatized oligonucleotides and NHS-activated beads. One may have attached and tested 6-12 sensors on beads in cross-reactive arrays, and be able to determine standard fingerprints for standard solutions and urine spiked with model (gluco)corticosteroids.

The first step would be to attach the sensors to beads. Some important differences in behavior may be observed between sensors on surfaces and in solution. First, in testing of initial batches of sensors (diastereomeric mixtures) one may see up to four-fold increase in signal on beads vs. only three-fold increase in solution, upon exposure to corticosteroids. This property may be attributed to the propensity of sensors to oligomerize in solution, which reduces the ability to sense ligands. Elimination of this process on beads is a (wanted) side effect of the dilution with an irrelevant oligonucleotide. However, one may have to study the influence of the structure of the irrelevant oligonucleotide on sensitivity of sensors, as one may noticed some differences between oligonucleotides. Second, sensitivity increased in the experiments with beads, and that much smoother dose-response curves were obtained. This property is a result of an increased number of measurements, i.e. characterization of each bead during flow cytometry is equivalent to a single solution measurement. In theory at least, counting of ten thousand beads would lead to almost 100-fold increase in sensitivity over a single measurement. In practice this number is smaller because of the lower sensitivity and precision (due to scattering) of bead counters in comparison to solution-phase luminometers. The use of a single, pure diastereomer may further improve sensors sensitivity. Further optimization process would likely lead to the main sensor having at least five-fold increase in fluorescence in response to corticosteroids, and one would expect to see a significant signal in the presence of 250 nM concentration of corticosteroid derivatives with 1,000 beads. Some sensors with electroneutral junctions have shown submicromolar sensitivity to steroids, and optimization of this process for these junctions may yield low nanomolar sensitivity for steroids—sufficient for analysis of some steroids in serum. Finally, there is batch-to-batch variability in regard to the dilution process; the use of diastereomerically pure sensors is essential to control this problem.

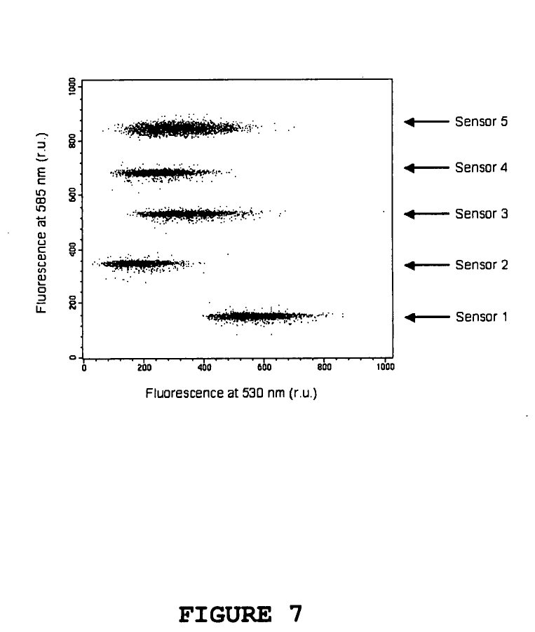

One may have a fully functional cross-reactive suspension array with at least six sensors. In order to read such an array, the beads must be encoded as to the sensor attached. One may use beads as one dimension and concentration of the second fluorescent color as another coding parameter. There are at least two options for encoding: to use commercial sources for fluorescently coded beads or to code beads during the dilution process with another irrelevant fluorescent oligonucleotide. One may favor using a commercial source, which would simplify the procedures for any group trying to reproduce this method. One may use streptavidin-coated microspheres commercially available from Polysciences, Inc. (Warrington, Pa.) and Bang Laboratories, Inc. (Fishers, Ind.). Beads produced by Polysciences may be used in optimization studies to determine condition of conjugation of sensors to beads and to develop initial protocols for analysis. One may have cross-reactive suspension arrays of eight (8) sensors. One may use 5×2 coding with five different concentrations of one fluorescent dye Starfire Red™ and two different sizes of beads commercially available from Bang Laboratories. Starfire Red™ is a fluorochrome with a very broad excitation band that allows it to be excited with all lasers used in flow cytometry technology. The dye emits mostly in the area longer then 670 nm (max 685 nm) with very little carry-out in the area of spectrum of fluorescein and phycoerythrin. These properties allow one to use fluorescein labeled sensors and open the possibility to use one more fluorochrome to encode further beads. One field may be reserved for unresponsive fluorescein-only labeled beads, as a positive control and standard, and the other for the negative control, containing unlabeled junction. One may attach eight different sensors to coded beads and mix all ten bead types together. These suspension arrays may be tested on standard solutions of steroids and spiked urine. A simulated example of expected results is shown in the FIG. 7. Each cross-reactive array of 10,000 beads (1,000 each) would likely provide a plot characteristic for the sample. One may test the following steroids to obtain characteristic fingerprints: corticosterone, testosterone 17-sulfate, deoxycorticosterone 21 -glucoside, dehydroisoandrosterone 3-sulfate, androstanediol 17-glucuronide, androstanone 3-glucuronide, deoxycholic, taurocholic, glycocholic acids and methylprednisolone sodium succinate (solumedrol). While there are examples in the literature of 8×8 arrays, allowing in principle the use of 62 sensors and two controls, based on preliminary results one may not need such an array. An example of a benchtop flow cytometer which may used is known as a FACS Calibur (BD Biosciences) which is equipped with two lasers, two detectors for light scatter, and four detectors for fluorescence.

Correlation of Hydrophobic Fingerprint of Urine with Disease States and Therapeutic Regimen

Correlation of Fingerprints with Disease States

The availability of hydrophobic receptors as a cross-reactive array, would allow one to rapidly move into further testing of the array, and practical applications. One may first validate the methodology on urine samples sent for determination of 17-ketosteroids and 17-hydroxytcorticosteroids by endocrinologists. In this way one would be able to compare whether fingerprints could substitute for values obtained through standard methods, and would be able to correlate fingerprints with specific disease states. Testing for these two groups of steroids (in combination with ACTH) can be used for diagnosing and differentiating Cushing syndrome (Table 1). Table 1 shows levels of 17OHCS and 17KS indicative of various forms of Cushing Syndrome. The levels indicated are in comparison to normal samples.

TABLE 1

| Differentiating | Cushing | Adrenal | Adrenal |
|---|---|---|---|
| Cushing Syndrome | Disease | Adenoma | Cancer |
| Urinary 17OHCS | High | High | High |
| Urinary 17KS | High | Low, Normal | Very High |

For example, a cortisol-producing adrenal adenoma is suggested if the urinary 17-OHCS is markedly elevated, while 17-KS is decreased or minimally changed from normal values. Adrenal carcinoma is suggested if both urinary 17-OHCS and 17-KS are strikingly elevated (New 1998, Elin 1996).

The range of values for these diagnostic tests is well within the sensitivity already displayed by proof-of-concept arrays (17-KS reference range 200-800 μM and 17-OH-CS 50-270 μM in 24-h urine). In preliminary results one may use diluted urine for this purpose, and it is possible that one would be able to read corticosteroids in serum as well. One of the most important immediate applications of arrays may be the screening for inborn errors of corticosteroid metabolism. For example, congenital adrenal hyperplasia, which occurs in 1:15,000 births, is characterized by overproduction of androgens. A complex multicomponent analytical procedure has been proposed to characterize infants with disorders of adrenal steroid production and excretion (Shackleton 1976). With arrays, one would be able to achieve simple and rapid detection of an exact defect, leading to a possible routine screening procedure. With larger arrays one would also be able to pick up fine differences in solubilizing groups and metabolites, which was not possible before without elaborate and impractical procedures. For example, fractionation of urinary 17-ketosteroids is reported to be an effective test in the evaluation of hirsutism (Shapiro 1989). While plasma and total urinary 17-KS were elevated in only 21% of the patients, elevated concentration of indivudual androsterone, etiocholanolone, and dehydroepiandrosterone were observed in 81% of the samples as determined by gas chromatography of hydrolysates. With the larger array one should be able to easily differentiate fingerprints of these mixtures without difficulty in a single step procedure.

Samples could be sorted according to age and gender. One may test, in a blinded fashion, specimens to establish fingerprints for the abnormal and normal values, as determined by standard tests. In this way, one could correlate the fingerprints with disease states. As carefully timed urine collection is a prerequisite for all excretory determinations, urinary creatinine level could be measured to determine the accuracy and adequacy of the collection procedure. The sensors operate well in buffered bodily fluids and one can obtain useful fingerprints from spiked urine.

Sample Collection

Aliquots (100 mL) of 24-h urine samples may be provided by a clinical chemistry core service at a hospital. A laboratory may handle between 10-15 24-h urine samples per week, and out of these approximately 1-2 per week may be sent for free cortisol determination (now preferred to classic 17-OHCS tests), and approximately 10 per year may be sent for 17-KS determination, and approximately 2 per year for 17-OHCS determination (pediatric endocrinology only). The samples may be handled by Quest Laboratories, except for pediatric endocrinology, which may be handled by Endocrine Sciences. The free cortisol is standardly determined by HPLC at Quest, while 17-KS and 17-OHCS are determined spectrophotometrically (Porter-Silber and modified Zimmerman procedures, respectively). Test interferences for these spectrophotometric tests come from a large number of substances (including almost any antibiotic), while fingerprints would not be affected by smaller molecules. Urine is preserved by addition of either boric or hydrochloric acid, and one would first have to neutralize all samples.

Samples could come from a diverse population, which could include a substantial percentage of women and minorities. Samples could be sorted according to age and gender. One could test, in a blind fashion, specimens to establish fingerprints for abnormal and normal values. One could also establish a feedback mechanism, wherein when a rule-out/rule-in diagnosis of Cushing's disease, adrenal adenoma or adrenal carcinoma is reported to the central lab, it will be forwarded. One could also request feedback in cases of Addison's disease, adrenogenital syndromes, pituitary insufficiency, castration, Klinefelter's syndrome, gout, myxedema, nephrosis and other disease states that could cause changes in the fingerprints. In this way, one could correlate the fingerprints with disease states.

Fingerprints may be sorted according to rule-in/rule-out diagnosis and one could analyze them to establish reference ranges for individual sensors which would be indicative of a disease state. Traditionally individual arrays are usually incorporated with neural networks and trained to recognize exemplary solutions of interest.

Sample Analysis

All samples sent for free cortisol, 17-KS and 17-OHCS determination be handled in following manner: One aliquot (12 mL) could be sent to LabCorp for 17-OHCS determination; other aliquot (25 mL) could be sent to LabCorp for 17-KS determination, while the third aliquot (15 mL) could be used to obtain fingerprints.

Fingerprinting could be performed by taking 250 μL aliquots of sterile filtered urine adding it to the 250 μL of neutralization buffer and 500 μL of binding buffer. With array with 9 sensors and 1 control, one could add a total of 10,000 beads to urine, and subject beads to fluorescence counting after one minute.

Correlation of Fingerprints with Therapeutic Regimen

Patients with systemic autoimmune disease, such as systemic lupus erythematosus (SLE), are frequently treated with oral or intravenous corticosteroids to achieve control of the immune system activation and inflammation that underlie the disease. Determination of appropriate steroid dose and route of administration is somewhat arbitrary, based on physician experience and patient response to therapy. In some cases, oral administration of corticosteroids is ineffective, while systemic administration of a comparable dose is clinically effective. An assay that would permit accurate and immediate assessment of available steroid concentration in urine or serum would permit improved patient management.

Many patients with SLE and many patients with rheumatoid arthritis (RA) receive corticosteroid therapy. After validating the efficacy of qualitative and quantitative detection of glucocorticoids spiked into urine, one could perform a study using serum and urine from such patients undergoing intravenous “pulse” steroid therapy. This therapy usually consists of 1 gram of methylprednisolone sodium succinate (solumedrol) administered intravenously daily for three days. One could collect a serum and urine specimen immediately prior to administration of each of the three doses of solumedrol, as well as immediately following infusion. A panel of sensor-encoded beads could be used to assay methyprednisolone and other steroid family molecules in these samples at each time point. The levels of steroids are predicted to increase after each administration of solumedrol. Data from the sensor panel assay could be correlated with standard biochemical assays for corticosteroids. Ultimately, one could conduct a study to correlate presence of methylprednisolone metabolites, as measured in the sensor panel assay, with clinical response to therapy.

Expansion of our Approach to Other Analytes

Coded beads could be used for lock-and-key sensors as well. While these sensors may be used to develop mechanistic intracellular probes, to use them for multiplexing with coded beads. However, the principles behind these assays are different from cross-reactive arrays.

Modified oligonucleotides for in vitro selection and amplification for analytical purposes and may result in cross-reactive arrays for other groups of molecules. For example, one selection process for recognition of lipids is based on using unnatural nucleotide benzoyl-aa-dUTP and it will yield a series of cross-reactive sensors capable of distinguishing shapes and lengths of fatty-acid side chains. Practical applications for such sensors will be in lipid analysis and detection of inborn errors of lipid metabolism. Some of these receptors will likely have smaller cavity sizes than three-way junctions, and one may test them for fingerprinting smaller molecules, including potential application in following metabolism of drugs. Another group of sensors may be based on aa-dUTP and could focus on detection of inositol phosphates. The third group focuses on oligosaccharides and contains low percentage of boronic acid derivatized nucleotides or a single derivative in a primer and aa-dUTPs. Cross reactive arrays on short peptides, may lead to an easy approach to rapid sequencing of peptides.

### REFERENCES

1. Albert K J, Lewis N S, Schauer C L, Sotzing G A, Stitzel S E, Vaid T P, Walt D R. Cross-reactive chemical sensor arrays. Chem Rev. 2000 Jul. 12;100(7):2595-626.
2. Axel R. The molecular logic of smell. Sci Am. 1995, 273, 1154.
3. Elin R J. Reference Intervals and Laboratory Values in “Cecil Textbook of Medicine” 1996.
4. Fidanza J A, Ozaki H, McLaughlin L W. Site-specific labeling of DNA sequences containing phosphorothioate diesters. J. Am. Chem. Soc.; 1992; 114(14); 5509-5517.
5. Ikeda H, Nakamura M, Ise N, Oguma N, Nakamura A, Ikeda T, Toda F, Ueno A. Fluorescent Cyclodextrins for Molecule Sensing: Fluorescent Properties, NMR Characterization, and Inclusion Phenomena of N-Dansylleucine-Modified Cyclodextrins J. Am. Chem. Soc.; 1996 118(45): 10980-10988.
6. Kato T, Yano K, Ikebukuro K, Karube I. Interaction of three-way DNA junctions with steroids. Nucleic Acids Res. 2000, 28(9): 1963-1968.
7. Kettman J R, Davies T, Chandler D, Oliver K G, Fulton R J. Classification and properties of 64 multiplexed microsphere sets. Cytometry 1998, 33(2): 234-43.
8. Lavigne J J, Anslyn E V. Sensing a paradigm shift in the field of molecular recognition: from selective to differential receptors. Angew. Chem. Int. Ed. 2001, 40(17), 3118-3130.
9. New M I. Inborn errors of steroidogenesis. Steroids 1998, 63(5-6), 238-242.
10. Nolan J P, Lauer S, Prossnitz E R, Sklar L A. Flow cytometry: a versatile tool for all phases of drug discovery. Drug Discov Today. 1999, 4(4), 173-180.
11. Nolan J P, Mandy F F. Suspension array technology: new tools for gene and protein analysis. Cell Mol Biol (Noisy-le-grand). 2001, 47(7), 1241-56.
12. Nolan J P, Sklar L A. Suspension array technology: evolution of the flat-array paradigm. Trends Biotechnol. 2002, 20(1), 9-12.
13. Rakow N A, Suslick K S. A colorimetric sensor array for odor visualization. Nature 2000, 406(6797), 710-713.
14. Schauer C L, Steemers F J, Walt D R. A cross-reactive, class-selective enzymatic array assay. J. Am. Chem. Soc. 2001, 123(38), 9443-9444.
15. Shapiro M S, Zelefsky S, Chayen R, Werber M M. Fractionation of urinary 17-ketosteroids by gas chromatography: a neglected procedure in the assessment of hyperandrogenicity in the hirsute female. J. Clin. Chem. Clin. Biochem. 1989, 27(1), 27-31.
16. Shackleton C H, Honour J W. Simultaneous estimation of urinary steroids by semi-automated gas schormatography. Investigation of neonatal infants and children with abnormal steroid synthesis. Clin. Chem. Acta. 1976, 69(2), 267-283.
17. Stojanovic M N, de Prada P, Landry D W. Homogenous Assays Based on Deoxyribozymes. Nucleic Acids Res 2000, 28, 2915-2918.
18. Stojanovic M N, de Prada P, Landry D W. Self-assembling aptameric sensors. J. Am. Chem. Soc. 2000, 122, 11547.
19. Stojanovic M N, de Prada P., Landry D W. Folding oligonucleotide-based sensors for cocaine. J. Am. Chem. Soc. 2001, 123, 4938.
20. Stojanovic M N, de Prada P, Landry D W. Catalytic Molecular Beacons. Chem. Bio. Chem. 2001, 2, 411-415.
21. Stojanovic M N, Landry D W. Aptamer-based calorimetric sensor for cocaine. J. Am. Chem. Soc. 2002 124, 9678.
22. Stojanovic M N, Green E G, Semova S, Landry D W. Cross-reactive Arrays Based on Three-Way Junctions. J. Am. Chem. Soc. 2003, 125, 6085-9.

We claim:

1. A suspension cross-reactive array for detecting the presence of at least one analyte, comprising:
a plurality of particles each encoded by a different selected characteristic;
a plurality of cross-reactive sensors, wherein at least one sensor is responsive to the presence of more than one different analyte, and wherein at least two sensors are responsive to the same analyte;
wherein each particle is attached to a different sensor, and wherein the identification of the analyte is detected by the arrangement of encoding of the particles' characteristics.

2. The array according to claim 1, wherein the selected characteristic is size.

3. The array according to claim 1, wherein the selected characteristic is fluorescence.

4. The array according to claim 1, wherein the selected characteristic is both size and fluorescence.

5. The array according to claim 1, wherein the cross-reactive sensors comprise oligonudeotides.

6. The array according to claim 1, wherein the selected characteristic is fluorescence and the cross-reactive sensors comprise oligonucleotides.

7. The array according to claim 1, wherein the sensors give fluorescent signals.

8. The array according to claim 5, wherein the sensors give fluorescent signals.

9. The suspension array according to claim 5, wherein decoding characteristics of particles and signals from sensors, attached to particles, measure simultaneously from each particle.

10. The array according to claim 9, wherein the identification of the analyte is detected using flow cytometry.

11. A method for detecting the presence of at least one analyte, comprising:
providing a plurality of particles each encoded by a different selected characteristic;
providing a plurality of cross-reactive sensors, wherein at least one sensor is responsive to the presence of more than one different analyte, wherein at least two sensors are responsive to the same analyte, and wherein each particle is attached to a different sensor;
detecting the identification of the analyte by the arrangement of encoding of the particles' characterstics.

12. The method according to claim 11, wherein the selected characteristic is size.

13. The method according to claim 11, wherein the selected characteristic is fluorescence.

14. The method according to claim 11, wherein the selected charactistic is both size and fluorescence.

15. The method according to claim 11, wherein the cross-reactive sensors comprise oligonucleotides.

16. The method according to claim 11, wherein the selected characterstic is fluorescence and the cross reactive sensors comprise oligonucleotides.

17. The method according to claim 11, wherein the sensors give fluorescent signals.

18. The method according to claim 15, wherein the sensors give fluorescent signals.

19. The method according to claim 7, wherein the identification of the analyte is detected by detecting the arrangement of encoding of the beads' characteristics simultaneously.

20. The method according to claim 19, wherein the identification of the analyte is detected using flow cytometry.
